(19) United States
(12) Patent Application Publication
(10) Pub. No.: US 2020/0188555 A1
(43) Pub. Date: Jun. 18, 2020

(54) PATCH FOR REGENERATING BIOLOGICAL TISSUES AND METHOD FOR PRODUCING SAME

(71) Applicant: VISCOFAN, S.A., Tajonar (ES)

(72) Inventors: Annika Huss, Frankenthal (DE); Silke Busch, Heidelberg (DE); Quintana Frigola Lluís, Heppenheim (DE); Prósper Cardoso Felipe, Cizur Menor (ES); Gavira Gómez Juanjo, Mutliva (ES); Rábago Juan-aracil Gregorio, Pamplona (ES)

(73) Assignee: VISCOFAN, S.A., Tajonar (ES)

(21) Appl. No.: 16/628,647

(22) PCT Filed: Jun. 26, 2018
(86) PCT No.: PCT/ES2018/070451

§ 371 Date: Jan. 3, 2020

(30) Foreign Application Priority Data
Jul. 3, 2017 (ES) P201730877

(51) Int. Cl. A61L 27/24; A61L 27/38; A61F 13/00

(52) U.S. Cl. CPC A61L 27/24; A61L 2430/40; A61F 13/00029; A61L 27/38

(57) ABSTRACT

Patch (1) for regenerating biological tissues in patients and method of manufacture associated with the same, comprising a collagen membrane (2) which comprises cells with a regenerative character; and at least one reinforcement element (3a, 3b) configured to increase the rigidity of said membrane (2), preventing the deformation of said membrane (2) during the handling thereof; wherein said reinforcement element (3a, 3b) can be a perimeter frame (3a) and/or a crossed attachment (3b) between the ends of said membrane (2); which provides a patch (1) which can be applied to any type of biological organ with maximum assurances of success as well as reducing the application time.

### TECHNICAL FIELD OF THE INVENTION

The present invention relates to a patch for regenerating biological tissues in patients and to the method of manufacturing the same; this patch belongs to the surgical and hospital sector.

The main object of the invention is that it is hygienically and effectively applied on one or several damaged organs of a patient such that said application enables a rapid regeneration of biological tissues owing to the regenerative capacity of the cells present in the patch itself and to the capacity thereof to help establish the biological fibre. It should also be pointed out how easy it is to manufacture, as well as the wide variety of geometric and morphological configurations which will to be obtained; wherein this wide variety is based on the site at which said patch object of the invention is going to be applied.

### BACKGROUND OF THE INVENTION

As an introduction, it is known that the use and application of patches for regenerating biological tissue in patients is being increasingly used wherein said patches are particularly a type of collagen sheet, currently called CCC; whereon controlled cell growth is effected such that a regenerative character of the patch itself is achieved. This extended use and application are due to the fact that in many surgical interventions, incisions and injuries are made on the organs of the patients with the aim of reaching the target area of the surgery. Said injuries involve a need for cicatrisation of said operated organs, which involves a long stay in a hospital room, in addition to the physical discomfort suffered by the patient. Furthermore, said injuries require complex cicatrisation, either due to the difficulty of the intervention or due to the very physiognomy of the patient, since they can have a lower number of cicatrising biological agents depending on the physical condition; this can involve a certain risk to the health of the patient.

Therefore, in order to reduce the cicatrisation time of the injuries caused and to reduce the risk of possible health problems in the patient, the use and application technique of CCC collagen patches is known for generating organ tissues comprising, as the name suggests, collagen protein sheets in charge of transporting material for the cells and converting into a substituted body membrane which degrades after a few weeks. Owing to this, the collagen is responsible for accelerating the regeneration and cicatrisation process of said injuries in the patient, thereby reducing the hospital stay and the risk of internal tearing due to the poor cicatrisation of the affected organs.

Even though it is very extensively used and the aim thereof of regeneration is appropriate for the previously mentioned advantages, it presents a significant disadvantage linked to how to apply it to the organs of the patients. This is due to the fact that the CCC collagen patches used currently have a deformable flexible tissue morphology which can be adapted to any type of surface according to the injury of the biological organ that is to be regenerated. However, this flexibility and adaptability is counterproductive when the surgeon responsible should introduce it and place it on said organ; surgeons usually handle sterilised surgical clamps that secure and grip, using one or several ends, the collagen patch and transport this patch to the affected organ such that the patch is folded and should be unfolded for suitable application which involves a delay in the surgery time and can also involve biological contamination of the patch since it must be unfolded and handled.

Therefore, given the disadvantages associated with the difficulties in applying patches for regenerating biological tissues in patients, it is necessary to find a new patch that can be applied on any type of surface of one or several biological organs that has a high adaptability and biological tissue-regeneration properties and which also facilitates and improves the application and placement techniques of said patch on the damaged organ and reduces the application time of said patch.

### DESCRIPTION OF THE INVENTION

The present invention relates to a patch for regenerating biological tissues in patients, comprising:
a collagen membrane comprising cells with a regenerative character; and
at least one reinforcement element configured to increase the rigidity of said membrane, preventing the deformation of said membrane during the handling thereof.

Wherein, for clarification purposes, it is observed that the membrane is made of a collagen material and which has a plurality of cells with a regenerative character for biological tissue since the method of regenerating said cells is carried out by growing said cells in the collagen membrane.

Therefore, thanks to the existence of said at least one reinforcement element, all the problems related to the step of applying the patch object of the invention on the organ of the patient are completely resolved; since this reinforcement element prevents the collagen membrane folding on itself, achieving a reduction in the application time of the patch from when it is taken from the sterilised container thereof to when it is applied to the organ of the patient.

In this respect, and in relation to said reinforcement element, different embodiment options are achieved, configured to increase, in any case, the rigidity of the collagen membrane connected to the patch object of the invention wherein:
said at least one reinforcement element comprises a perimeter frame with respect to said membrane such that it prevents the bending of the patch since the preferred securing by the surgeon with respect to the patch is carried out along the perimeter contour of the same and, given that it is reinforced. it makes the easy and effective folding over on itself more difficult.
said at least one reinforcement element comprises a crossed attachment between the ends of said collagen membrane; once again increasing the rigidity of the entire patch in a manner similar to a triangular bar structure which results in the non-deformability of the patch and can be complemented by the described perimeter frame which ensures the advantage of the application time mentioned above.

Furthermore, with the aim of simplifying the design and manufacture of the patch object of the invention, the preferred option is considered the one wherein said at least one reinforcement element is manufactured with the same material as said collagen membrane. This also ensures that there are no incompatibilities between the materials forming said patch which is a homogenous material and increases the guarantee of acceptance and reabsorption of the patch with respect to the biological organ of the patient.

Moreover, the method of manufacturing the patch object of the invention is described, comprising the following steps:

a) manufacturing a collagen membrane;

b) manufacturing said reinforcement element;

c) placing said at least one reinforcement element with respect to said membrane;

d) sterilising the membrane; and

e) adding cells with a regenerative character on said membrane.

Wherein the aforementioned steps involve an easy and logical method of manufacture which does not involve a substantial increase to the cost compared to the existing prior art; which can have different possibilities of manufacture with respect to how to design said at least one reinforcement element.

It should be pointed out that, since said patch falls within the surgical sector, the preferred option is the one wherein the method of manufacturing the patch comprises, subsequent to step c) and prior to step e), sterilising the assembly formed by the collagen membrane and said at least one reinforcement element; ensuring hygienic and healthy use in the patient affected.

With respect to the reinforcement element, it can be made of an element external to the collagen membrane and it is described how in step c), the placement of said at least one reinforcement element with respect to said membrane is carried out by means of adhering two elements with water, for example. Or an option may be considered wherein and during step c) the placement of said at least one reinforcement element with respect to said membrane is carried out in two sub steps:

c1) wetting the membrane, placing the reinforcement; and

c2) irradiating the membrane and the reinforcement element by means of gamma radiation.

Furthermore, in contrast to the use of a reinforcement element external to the membrane; the preferred option is described wherein the steps a) and b) of manufacturing the membrane and said at least one reinforcement element are carried out in a single step since said at least one reinforcement element is part of the membrane itself, and both entities being collagen; and wherein the step c) of placing said at least one reinforcement element with respect to said membrane is carried out by bending at least one of the borders of said membrane. This simplifies the manufacture thereof and ensures the homogeneity of the collagen patch material thus obtained.

In a similar manner, and which can be considered complementary or independent embodiments, the option is described wherein the steps a) and b) of manufacturing the membrane and said at least one reinforcement element are also carried out in a single step, since said at least one reinforcement element is part of the collagen membrane itself, but wherein said step c) of placing said at least one reinforcement element with respect to said membrane is carried out by bending at least the internal surface of said membrane.

Therefore, with the proposed invention, a patch is obtained for regenerating biological tissues in patients which can be applied to any type of biological organ with maximum guarantees of success, reducing the application time; and it also highlights the easy manufacture of the same and the wide variety of geometric shapes to be implemented according to the location at which said patch object of the invention will be applied.

### DESCRIPTION OF THE DRAWINGS

In order to complement the description being given and with the aim of aiding a better understanding of the features of the invention, according to a preferred exemplary embodiment of the same, as an integral part of said description, a drawing is included wherein the following has been represented in an illustrative and non-limiting manner:

FIG. 1 shows a three-dimensional view of the patch for regenerating biological tissues in patients object of the invention, being ensured by surgical material.

### PREFERRED EMBODIMENT OF THE INVENTION

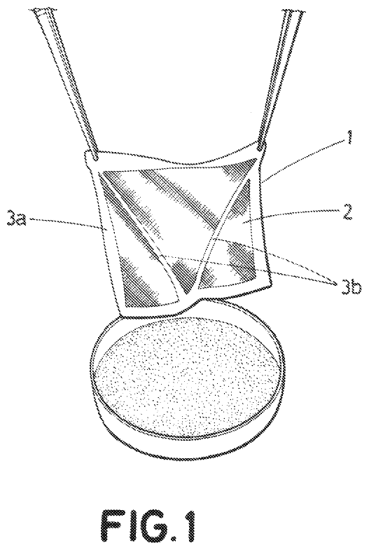

FIG. 1 shows a patch (1) for regenerating biological tissues in patients object of the invention comprising:
a collagen membrane (2) with a plurality of cells with a regenerative character; and
two reinforcement elements (3a, 3b), each one configured to increase the rigidity of said membrane (2), preventing the deformation of said membrane (2) during the handling thereof.

Said reinforcement elements (3a, 3b) are defined by a perimeter frame (3a) with respect to said membrane (2); and by a pair of crossed attachments (3b) between the ends of said membrane (2); which increase the structural rigidity of the entire patch (1) in a highly effective manner since said reinforcement elements (3a, 3b) are manufactured with the same collagen material as said membrane (2), simplifying to a large extent the installation process thereof during surgery.

In this respect, the preferred methods of manufacture of the patch (1) for regenerating biological tissues in patients, comprise the following steps:
a) manufacturing the collagen membrane (2) and the reinforcement elements (3a, 3b);
b) placing and generating said reinforcement elements (3a, 3b) with respect to said membrane (2). In which the step b) of placing said reinforcement elements (3a, 3b) with respect to said membrane (2) is carried out by bending each one of the borders of said membrane (2) in order to implement the perimeter frame (3a); and each one of the crossed attachments (3b) is carried out by folding a portion of the internal surface of said membrane (2);
c) sterilising the assembly formed by the membrane (2) and the reinforcement elements (3a, 3b); and lastly
d) adding cells with a regenerative character on said membrane (2).

In light of the present description and figure, a person with skill in the art could understand that the embodiments of the invention described can be combined in multiple manners within the object of the invention. The invention has been described according to some preferred embodiments of the same, but for the person with skill in the art it will be evident that there can be multiple variations made in said preferred embodiments without departing from the object of the claimed invention.

1. A patch (1) for regenerating biological tissues in patients comprising a collagen membrane (2) which comprises cells with a regenerative character and is characterised in that it also comprises a reinforcement element (3a, 3b) configured to increase the rigidity of said membrane (2), preventing the deformation of said membrane (2) during the handling thereof.

2. The patch (1) according to claim 1, characterised in that said at least one reinforcement element (3a, 3b) comprises a perimeter frame (3a) belonging to said membrane (2).

3. The patch (1) according to any of the preceding claims, characterised in that said at least one reinforcement element (3a, 3b) comprises a crossed attachment (3b) between ends of said membrane (2).

4. The patch (1) according to any of the preceding claims, characterised in that said at least one reinforcement element (3a, 3b) is manufactured with the same material as said membrane (2).

5. A method of manufacturing a patch (1) for regenerating biological tissues in patients, characterised in that it comprises the following steps:
a) manufacturing a collagen membrane (2);
b) manufacturing at least one reinforcement element (3a, 3b);
c) placing said at least one reinforcement element (3a, 3b) with respect to said membrane (2);
d) sterilising the membrane; and
e) adding cells with a regenerative character on said membrane (2).

6. The method of manufacturing a patch (1) according to claim 5, characterised in that subsequent to step c) and prior to step e), the assembly formed by the collagen membrane (2) and said at least one reinforcement element (3a, 3b) are sterilised.

7. The method of manufacturing a patch (1) according to any of claims 5 and 6, characterised in that in step c), placing said at least one reinforcement element (3a, 3b) with respect to said membrane (2) is carried out in two sub steps:
c1) wetting the membrane (2); and
c2) irradiating the membrane (2) and the reinforcement element (3a, 3b) by means of gamma radiation.

8. The method of manufacturing a patch (1) according to any of claims 5 and 6, characterised in that the steps a) and b) of manufacturing the membrane (2) and said at least one reinforcement element (3a, 3b) are carried out in one single step since said at least one reinforcement element (3a, 3b) is part of the membrane (2) itself and both entities being collagen; and wherein the step c) of placing said at least one reinforcement element (3a, 3b) with respect to said membrane (2) is carried out bending at least one of the borders of said membrane (2).

9. The method of manufacturing a patch (1) according to any of claims 5 to 8, characterised in that steps a) and b) of manufacturing the membrane (2) and said at least one reinforcement element (3a, 3b) are carried out in one single step since said at least one reinforcement element (3a, 3b) is part of the membrane (2) itself and both entities being collagen; and wherein the step c) of placing said at least one reinforcement element (3a, 3b) with respect to said membrane (2) is carried out by folding at least a portion of the internal surface of said membrane (2).
